(19) United States
(12) Patent Application Publication
(10) Pub. No.: US 2016/0184045 A1
(43) Pub. Date: Jun. 30, 2016

(54) METHOD FOR PRODUCTION OF LOCAL ILLUMINATION PATCH CORD AND CORRESPONDING PATCH CORD

(71) Applicant: FAMOLDE - FABRICAÇÃO E COMERCIALIZAÇÃO DE MOLDES, S.A., Marinha Grande (PT)

(72) Inventors: Joaquim HENRIQUES MARTINS, MARINHA GRANDE (PT); Bruno DINIS MARTINS, MARINHA GRANDE (PT); Pedro Jorge FERREIRA OLIVEIRA, MARINHA GRANDE (PT); Marco Filipe DIAS, MARINHA GRANDE (PT); Carlos Alberto DUARTE PINHO, MARINHA GRANDE (PT)

(21) Appl. No.: 14/904,436

(22) PCT Filed: Feb. 13, 2014
(86) PCT No.: PCT/PT2014/000010

§ 371 Date: Jan. 12, 2016

(30) Foreign Application Priority Data
Jul. 12, 2013 (PT) 107061

(51) Int. Cl. A61B 90/30; G02B 6/04; F21V 8/00

(52) U.S. Cl. CPC A61B 90/30; G02B 6/0008; G02B 6/0006; G02B 6/04; A61B 2090/306

(57) ABSTRACT

Present invention discloses a method of producing a local illumination patch cord and the corresponding patch cord, based on the technology of a multi-fiber optic cable, from the entrance of supplies till the final product—the local illumination patch cord. Among various available cables, none of those on which tests were carried out met the following final product requirements: flexibility, light efficiency, and decrease of factors affecting its resistance, namely the heat. In opposition to the current accessories available in the market, present invention decentralizes the thermal filter from the cable to the coupling, enabling heat dissipation before entering the illumination cable. Regarding the luminous efficiency, present invention defines a technique with a more effective manner of aggregating the multi-fibers of the cable, with significant savings in production times, and raises polishing quality to the highest level of light transmission.

### TECHNICAL FIELD AND STATE OF THE ART OF THE INVENTION

The scope of present invention relates to the field of optical illumination, with particular focus on the medical sector as auxiliary device in surgery.

In the current market there are numerous applications for optical fiber, but there are few available techniques for the design of fiber-optic cables for illumination. Among the various available cables, none meets such requirements as: flexibility, light efficiency and decrease of factors affecting its resistance, namely the heat.

In opposition to the current accessories available in the market, present invention decentralizes the thermal filter from the cable to the coupling, enabling heat dissipation before entering the illumination cable (1). Regarding the luminous efficiency, present invention defines a technique with a more effective manner of aggregating the multi-fibers of the cable (1), significantly saving production times, and raises polishing quality to the highest level of light transmission.

### BRIEF DESCRIPTION OF THE INVENTION

Present invention reflects the need of having a product that incorporates a particular medical device and allowing it to have autonomous qualities of flexibility, reduced volume, high capacity of light transmission, longevity and low cost.

Since it was not found in the prior art a product with such requirements, present invention has been developed with a reduced transmission section, but still with a very high capacity of carrying light, equivalent to similar devices with twice the cross-sectional area. To this end, PMMA (Poly-methylmethacrylate) fibers where used in alternative to the existing glass and liquid fibers. In fact, the PMMA fibers provide a higher quality both at the structural level—they do not break so easily—and at the level of purity—a cooler white light—containing a low degree of impurities in its composition. A set of 19 fibers with a diameter of 0.0005 m was aggregated and coated by a flexible crystal PVC sleeve (which in turn provides protection and flexibility to the patch cord), thus maximizing the flow of light in a reduced section of 0.0025 m using a UV curing liquid adhesive.

This adhesive provides a very low curing time compared to an adhesive based on epoxy resin, and a very satisfactory fixation quality without damage of the fibers by either chemical reaction or by melting them through dry heat.

To ensure the quality of the transmitted light, and after testing several techniques including sandpaper of different grit sizes, diamond pastes and other polishing products existing on the market, it was found that the most effective method, fast and less expensive, would be the use of polishing soap. Therefore, after aggregation of the fibers the patch cord is submitted to a rectification process of the same (thus eliminating surplus) in a fine-grained grinding wheel (11), and subsequently in a felt buffing wheel (12) with a layer of soap, thus carrying out an action of wear and friction, respectively, at an averaged radial rotation.

In order to improve the longevity of the device, traditionally conditioned by fiber overheating and hence deterioration thereof, the thermal filter of the patch cord was decentralized to the coupling that accommodates the patch cord in each type/brand of light source commercially available.

### DESCRIPTION OF THE FIGURES

FIG. 1—Scheme of the Local Illumination Patch Cord.

FIG. 2—Module of Ultraviolet (UV) Exposure.

FIG. 3—Support of the Patch Cords for Exposure.

FIG. 4—Module of Fiber Polishing.

FIG. 5—Storz coupling with built-in thermal filter.

FIG. 6—Storz coupling with built-in thermal filter—cross-sectional view.

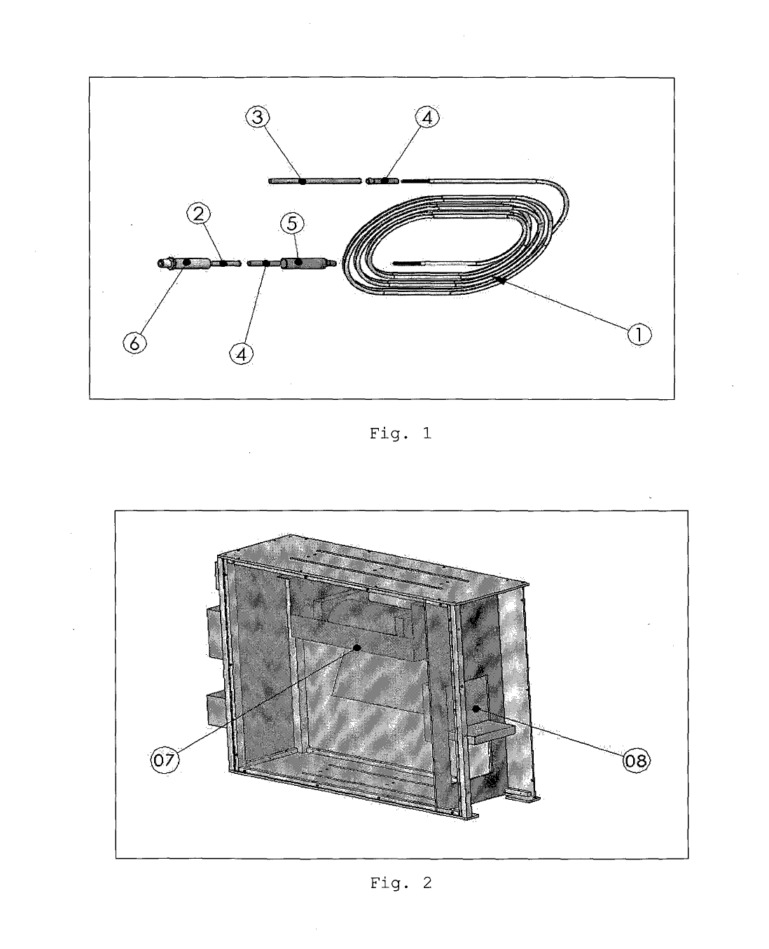

### LIST OF REFERENCE NUMBERS

1. Multi-fiber optic cable (19×ø0.5), coated with flexible crystal PVC sleeve

2. 28 mm tubular rivet

3. 88 mm tubular rivet

4. ø4 shrinkable sleeve

5. ø9 shrinkable sleeve

6. Optic connector

7. UV lamp

8. Door

9. Spring clip;

10. Polishing machine

11. Fine-grained grinding wheel

12. Felt buffing wheel

13. Polishing support

14. Guide bar

15. Knob

16. Base of the polishing support sliding in the guide bar

17. Coupling

18. Thermal filter

19. O-ring

### DETAILED DESCRIPTION OF THE INVENTION

Present invention discloses a method for producing a cable (1) for local illumination, which consists of a set of steps aided by the following modules:

The ultrasonic module has the function to ensure the cleaning of metal components in order to maintain the final device free of residues;

The Ultraviolet (UV) exposure module is intended to irradiate UV light, aiming the curing process in the liquid adhesion of the fibers to the metallic terminal;

The polishing module has the functionality of performing the final polishing of the fiber surface, so that it is free from any influent light barrier. The module is semi-automatic and has two devices: one for rectification and another for final polishing;

The thermal-sealing is a semi-automatic system which has the function of sealing the device packing with a sterilizable sealing paper.

The steps constituting present invention are:

### A. Preparation of Cable Terminals:

Cleaning metallic parts: tubular rivets (2) (3) and optic connector (6)—this step should occur when receiving metallic parts, thereby allowing shortening of the illuminators production cycle time;

Drying metallic parts—after the cleaning cycle and washing of the components with flowing water, metallic parts should be dried with compressed air;

### B. Cutting and Installation of Tubular Rivets in the Fiber:

Cutting the cable, which must be cut into pieces of approximately 3 m using a precision cutting pliers and should then be stripped in a portion of approximately 0.1 m at one end and of 0.04 m at the other end;

Placing the metallic rivet with size 0.088 m (3) in the 0.1 m stripped end, with the respective head oriented to the cable side;

Placing the metallic rivet with size 0.028 m (2) in the other end, similarly to the aforementioned manner;

Adjustment of each metallic rivet between the rivet head and the flexible PVC sleeve;

Note: the fibers should be aligned on the rivet, so they are not intertwined.

### C. Gluing—Application of UV Adhesive and UV Exposure (This Step Takes Place in the UV Exposure Module):

Placing the patch cord with the two rivets in the UV exposure support, fixing them with the spring clips (9);

Placing up to 3 patch cords in the support;

Trimming to an average length of 0.01 m the ends of each fiber passing the rivet head;

Application on each end of the fiber (passing each rivet), with the UV adhesive applicator resting on the tip and pushing the plunger until leaving a small concave drop in the base of the fiber tip;

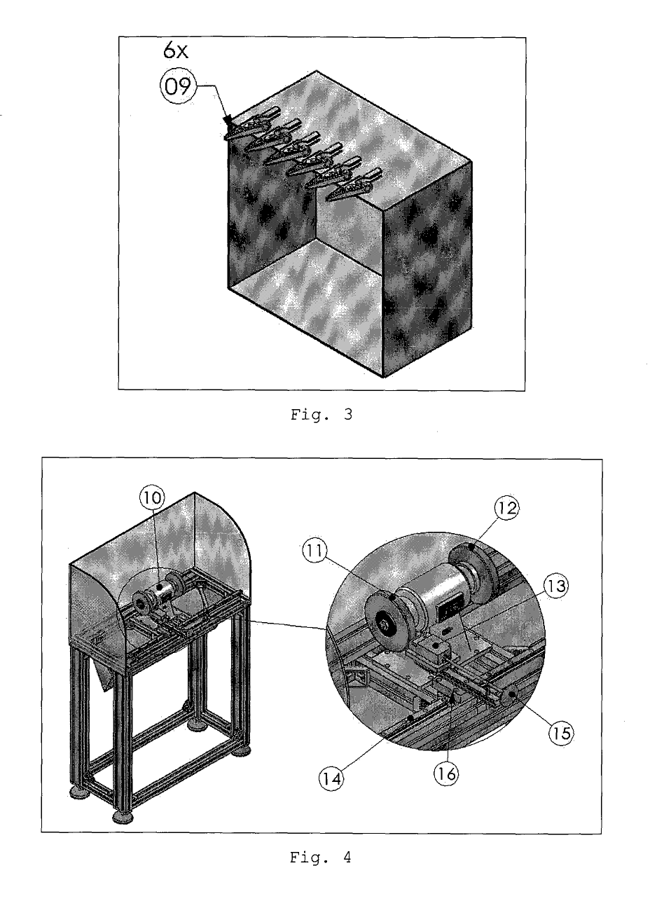

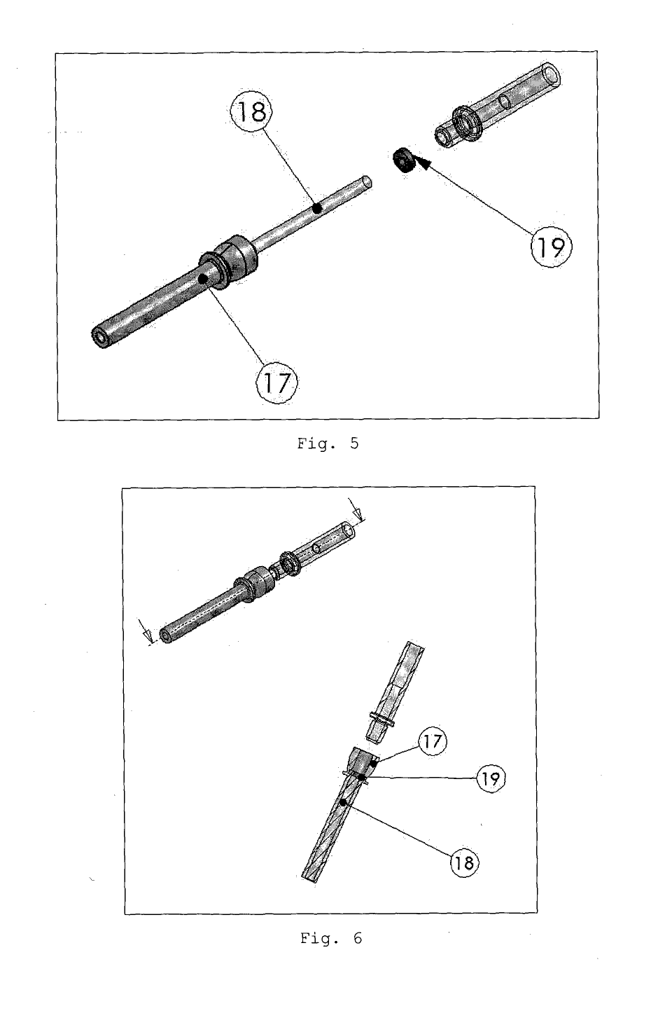

UV exposure—UV exposure is made within the UV exposure module (FIG. 2), in which exists a UV lamp (7) of approximately 1500 W, opening the door (8) and placing the support (FIG. 3) with the cables, whose tips are oriented upwards, during a predetermined time. This way, the production time is substantially reduced and hence the cost of the final product, which would not be possible when an epoxy adhesive or fusion method is used requiring curing and heating times of around 24 hours and 30 minutes, respectively.

### D. Fiber Polishing

The metallic tips of the patch cord should be placed in the support (13) of the polishing module;

Adjusting the head of each rivet on the back surface of the support;

Turning on the polishing machine (10) presenting the rectification and polishing wheels;

Rectification of the tips with the fine-grained grinding wheel (11) while advancing the support by turning the knob (15);

After rectification, sliding the support on the guide bar (14) to the felt buffing wheel (12) with soap along the base of the polishing support (16), and proceeding with polishing for about 30 seconds;

Cleaning with a dry cloth;

### E. Quality Control:

Cleaning the polished tips with gauze soaked in solvent (ethanol), followed by a dry gauze;

Turning on the light source, connecting the patch cord to it and to the support of the luxmeter;

Reading the amount of light (in lux);
if the reading does not show values above 300,000 lux, repeat the polishing procedure again;
if after 3 attempts the desired values are not obtained, reject the cable;

### F. Mounting of the Optic Connector and Applicable Optical Device:

Application of a 0.03 m portion of ø4 shrinkable sleeve (4) at each end of the cable, covering the flexible PVC coating and partially the tubular rivet (3). The shrinkable sleeve will confer protection to the fibers when handling the patch cord, because it is an area more susceptible to breakage; alternatively to the use of a shrinkable sleeve it can be used liquid silicone molded to the configuration of terminals versus fiber cable (1);

Retracting the sleeve (4) with the help of a hot air station;

Application of a 0.05 m portion of ø9 shrinkable sleeve (5) at the end of the cable (1) with the rivet of 0.028 mm (2), placing afterwards a small drop of adhesive (cyanoacrylate) on the rivet and making it slide about the optic connector (6) till the head of the rivet internally abuts. The application of cyanoacrylate adhesive serves to ensure that the terminal does not move within the optic connector and consequently reduce the light transmission capacity of the patch cord;

Adjusting the ø9 shrinkable sleeve (5) on the collar of the connector and retract it, thus adjusting the cable (1) to the connector;

Placing the applicable optical device at the opposite end of the cable;

The thermal filter is a part of the cable itself in the case of cables currently available in the market but, for improving its longevity, the thermal filter (18) is here applied in the coupling, for instance a Storz type (17), thus decentralizing the overheating factor to the coupling and preventing early destruction of the fibers when connected to a light source;

The coupling includes the thermal filter (18) followed by an O-ring (19) which prevents the filter from rolling out of the coupling. Since there are different types/brands of light sources, the couplings are supplied separately from the patch cord, thus allowing the end user to select and apply the coupling that best matches their use.

### G. Packing of Illuminator and Package Sealing:

Preparing the thermal sealer;

Turning on the thermal sealer by turning the on/off button located on the left side of the machine;

Confirming availability of sealing film to seal the packages;

Turning on the thermal sealer system by pressing the “on” button;

Waiting until the system reaches the desired temperature;

Pressing the two green buttons simultaneously and holding under pressure until the sealant mold enters the sealing area;

Waiting for the sealing process to take place;

Removing the packages already sealed after the sealing cycle ends;

Placing the cable (1) of the illuminator in the thermoformed package, being the applicable optical device housed in the “box” of the package periphery and the optic connector in the inner “box” of the package;

Placing the retaining clips on respective latches after packaging the cable (1) in the package;

Placing the package in the thermal sealer tray with the package “head” oriented upward;

Waiting the thermal sealer reaches the desired temperature (122 ° C.);

Starting the sealing;

Pressing the two green buttons simultaneously and holding under pressure until the sealant mold enters the sealing area;

Waiting for the sealing process to take place;

Removing the packages already sealed after the sealing cycle ends;

Printing the labels with information relating to the lot;

Application of the label on the sealing paper, in the respective placing area;

Placing the packages in card boxes of 5 units each.

### H. Finalization of the Lot:

The lot number is assigned to the quantity produced in a production cycle, so it may have different quantities. As such, the printed form that records all the history of the lot accompanies each lot of each production cycle.

The final lot for sending to sterilization is composed of the volume that occupies a Euro-pallet. The 5 units boxes are packed in corrugated boxes (14×5 units). The Euro-pallets are lined with plastic wrap and send to the sterilizer.

The product obtained by present invention method is a patch cord with the following technical features:

Terminals;

Connection or connector;

19 PMMA optical fibers with 0.0005 m diameter, coated with a 0.0025 m flexible crystal PVC sleeve (0.0025 m refers to the inner diameter of the fiber, the outer diameter being 0.0045 m) using an ultraviolet curing liquid adhesive;

The ends of the fiber pass each rivet in an average length of 0.01 m;

Ultraviolet curing adhesive on each end of the fiber;

0.03 m of shrinkable sleeve (4) at each end of the cable, covering the flexible PVC coating and partially the tubular rivet (3);

The cable end with the 0.028 mm rivet (2) presents a 0.05 m portion of shrinkable sleeve (5) and adhesive over the rivet;

The opposite end of the cable presents the applicable optical device;

The coupling includes the thermal filter (18) followed by an O-ring (19).

The previous description for a preferred embodiment of the invention is presented for purposes of illustration and description and is not intended to limit the invention to the form disclosed, being possible modifications and variations in accordance with the above description.

For instance, the polishing method may be replaced by other methods whose results reach or exceed the levels of light transfer of the current method, such as an automatic equipment existing in the market constituted by a unit that comprises three distinct stages: polishing, cleaning and inspection, where the polishing and cleaning are subdivided into three substations, progressively maximizing the polishing and cleaning levels, respectively, and the visual inspection gives greater accuracy in cable quality.

1. Method for production of local illumination patch cords with the following steps:
a) Cleaning and drying of tubular rivets (2) (3) and optic connector (6);
b) Cutting and assembling of tubular rivets (2) (3) in the fiber;
c) Application of the ultraviolet curing adhesive;
d) Ultraviolet exposure;
Characterized by the
e) Polishing the fibers with a rectification of the ends is made in the fine-grained grinding wheel (11) followed by sliding the support on the guide bar (14) to the felt buffing wheel (12) with soap, and respective polishing for 30 seconds;
f) Assembling the optic connector and applicable optical device;
g) Application of the thermal filter (18) in the adapter (17), followed by an O-ring (19).

2. Method according to claim 1, characterized, in that the ultraviolet exposure process is performed with a UV lamp (7) of about 1500 W.

3. Method according to claim 1, characterized in that the assembling procedure of the optic connector (6)
and—of the applicable optical device comprises the following steps:
a) Application of a 0.03 m portion of shrinkable sleeve (4) at each end of the cable, covering the flexible FVC coating and partially the tubular rivet (3);
b) Retracting the sleeve (4) with the help of a hot air station;
c) Placing of a 0.05 m portion of shrinkable sleeve (5) at the end of the cable presenting the rivet (2);
d) Placing of an adhesive on the rivet (2) and respective sliding about the optic connector (6) till the head of the rivet internally abuts;
e) Adjusting the shrinkable sleeve (5) on the collar of-the connector and retracting it, thus adjusting the cable to the connector;
f) Placing the applicable optical device at the opposite end of the cable;
g) Application of the thermal filter (18) in the adapter followed by an O-ring (19).

4. Patch cord obtained by the method of claim 1,
characterised in that it has:
a) Terminals;
b) Connection or connector;
c) 19 PUMA optical fibers with 0.0005 m diameter, coated with a 0.0025 m flexible crystal FVC sleeve using an ultraviolet curing liquid adhesive;
d) The ends of the fiber pass each rivet in an average length of 0.01 m;
e) Ultraviolet curing adhesive on each end of the fiber;
f) 0.03 m of shrinkable sleeve (4) at each end of the cable, covering the flexible pvc coating and partially the tubular rivet (3);
g) The cable end with the 0.028 mm rivet (2) presenting a 0.05 m portion of shrinkable sleeve (5) and adhesive over the rivet;
h) The opposite end of the cable presenting the applicable optical device;
i) The adapter including the thermal filter (18) followed by an O-ring (19).

5. Patch cord obtained by the method of claim 2, characterised in that it has:
a) Terminals;
b) Connection or connector;
c) 19 PUMA optical fibers with 0.0005 m diameter, coated with a 0.0025 m flexible crystal FVC sleeve using an ultraviolet curing liquid adhesive;
The ends of the fiber pass each rivet in an average length of 0.01 m;
e) Ultraviolet curing adhesive on each end of the fiber;
f) 0.03 m of shrinkable sleeve (4) at each end of the cable, covering the flexible PVC coating and partially the tubular rivet (3);
g) The cable end with the 0.028 mm rivet (2) presenting a 0.05 m portion of shrinkable sleeve (5) and adhesive over the rivet;
h) The opposite end of the cable presenting the applicable optical device;
l ) The adapter including the thermal filler (16) followed by an Q-ring (19).

6. Patch cord obtained by the method of claim 3, characterised in that it has:
a) Terminals;
b) Connection or connector;
c) 19 PUMA optical fibers with 0.0005 m diameter, coated with a 0.0025 m flexible crystal FVC sleeve using an ultraviolet curing liquid adhesive;
d) The ends of the fiber pass each rivet in an average length of 0.01 m;
e) Ultraviolet curing adhesive on each end of the fiber;
f) 0.03 m of shrinkable sleeve (4) at each end of the cable, covering the flexible pvc coating and partially the tubular rivet (3);
g) The cable end with the 0.028 mm rivet (2) presenting a 0.05 m portion of shrinkable sleeve (5) and adhesive over the rivet;
h) The opposite end of the cable presenting the applicable optical, device;
i) The adapter including the thermal filter (16) followed by an O-ring (19).

7. Patch cord obtained, by the method of claim 4, characterised in that it has:
a) Terminals;
b) Connection or connector;
c) 19 PUMA optical fibers with 0.0005 m diameter, coated with a 0.0025 m flexible crystal FVC sleeve using an ultraviolet curing liquid adhesive;
d) The ends of the fiber pass each rivet in an average length of 0.01 m;
e) Ultraviolet curing adhesive on each end of the fiber;
f) 0.03 m of shrinkable sleeve (4) at each end of the cable, covering the flexible pvc coating and partially the tubular rivet (3);
g) The cable end with the 0.028 mm rivet (2) presenting a 0.05 m portion of shrinkable sleeve (5) and adhesive over the rivet;
h) The opposite end of the cable presenting the applicable optical device;
i) The adapter including the thermal fitter (18) followed by an O-ring (19).
